(19) United States
(12) Patent Application Publication
(10) Pub. No.: US 2008/0091226 A1
(43) Pub. Date: Apr. 17, 2008

(54) MICRONEEDLE DEVICE

(75) Inventors: Yehoshua Yeshurun, Haifa (IL); Yotam Levin, Nes Ziona (IL); Yotam Almagor, Jerusalem (IL); Gilad Lavi, Rishon Letzion (IL); Meir Hefetz, Mizpe Harashim (IL); Yoel Sefi, Malkia (IL)

Correspondence Address: DR. MARK M. FRIEDMAN;C/O BILL POLKINGHORN - DISCOVERY DISPATCH, 9003 FLORIN WAY, UPPER MARLBORO, MD, 20772, US

(73) Assignee: Nanopass Technologies Ltd., Haifa (IL)

(21) Appl. No.: 11/549,982

(22) Filed: Oct. 17, 2006

(51) Int. Cl. A61B 17/34

(52) U.S. Cl. 606/186

(57) ABSTRACT

A microneedle device for delivery or sampling of fluids to or from intradermal layers of the skin of a mammalian subject includes a skin contact configuration defining an orientation of the device and a number of microneedles The microneedles are deployed relative to the skin contact configuration so that, when deployed against the skin, a first region of a peripheral surface of the microneedle is deployed roughly parallel to the initial plane of the skin. A fluid flow bore intersects the first region of the peripheral surface.

### FIELD AND BACKGROUND OF THE INVENTION

The present invention relates to microneedle devices and, in particular, it concerns a microneedle device for drug delivery or diagnostic sampling with geometry which minimizes delivery depth and skin deformation during insertion.

Of particular relevance as background to the present invention are PCT patent application publication no WO 2005/049107 A2 and U.S. patent application publication no 2005/0209566, both commonly assigned with the present invention, which are hereby incorporated by reference in their entirety. These documents disclose a system and method for delivering fluid into a flexible biological barrier employing a microneedle structure wherein a final position of microneedles inserted into the biological barrier is generally sideways projecting from the delivery configuration instead of the conventional downwards projecting arrangement. This technique is referred to herein for convenience as “side insertion.” The microneedles project from a relief surface which is distinct from a primary biological-barrier contact region of the delivery configuration, and is typically angled upwards so that it is not in face-on relation to the biological barrier. During insertion, the contact region is brought into contact with the biological barrier and moved parallel to the surface of the flexible biological barrier so as to generate a boundary between a stretched portion and a non-stretched portion of the barrier. Typically concurrently with this motion, the microneedles penetrate into the flexible biological barrier such that, at the end of the motion, the microneedles extend into the flexible biological barrier from the boundary region in a direction towards the non-stretched portion. Fluid is then injected through the bores of the hollow microneedles towards the non-stretched portion.

A schematic representation of a device for implementing the system and method of the aforementioned documents is shown in FIGS. 1A and 1B. In this case, a rectangular block is provided with a linear array of microneedles spaced along the lower edge of one face adjacent to the corner where that face intersects the skin contact surface. The design shown employs orthogonal flow channels perpendicular to faces of the block. As a result, the flow channel structure requires two perpendicular channels passing through a major part of a corresponding dimension of the block in order to provide a horizontal outlet channel which is not immediately adjacent to the lower extremity of the block.

Preferred microneedle designs for implementing the aforementioned device are structures similar to those disclosed in U.S. Pat. No. 6,533,949, also co-assigned with the present invention, which is hereby incorporated by reference in its entirety The needles described therein have a generally triangular cross-sectional shape including one or more upright wall intersecting with a sloped surface (referred to below as the “bevel surface” of the needle) through which a fluid flow channel passes. This needle structure lends itself to two distinct implementations of the above-mentioned device, as illustrated here in FIGS. 2A and 3A, corresponding to FIGS. 11j and 11h of WO 2005/049107 A2, respectively.

Specifically, referring to FIG. 2A, this shows a microneedle device configuration according to the teachings of the aforementioned U.S. 2005/0209566 in which the bevel surface is face down. This configuration in combination with an insertion motion similar to that described in the aforementioned US application is highly effective at penetrating and retaining engagement with the skin, and has low flow impedance for injection into the skin. Nevertheless, it suffers from certain disadvantages. Specifically, as illustrated in FIG. 2B, the skin passing over the needle is highly stretched along the entire length of the needle, applying significant stress to the thin tip of the needle and causing significant trauma to the tissue. Furthermore, depending upon the depth of initial penetration of the needles, the device may lift a significant ruck of skin, resulting in injection of fluids to relatively deep layers of the dermis.

An alternative approach proposed in WO 2005/049107 A2 is illustrated in FIGS. 3A and 3B. In this case, the needles are deployed bevel-up. This change of geometry greatly reduces the stretching of skin near the point of the needle, thereby reducing stress on the needle tip and trauma to the skin tissue In practice, however, this approach is problematic due to the high flow impedance introduced by the stretched epidermal tissue overlying the outlet aperture.

There is therefore a need for a microneedle device which would provide reliable shallow drug delivery or diagnostic sampling while minimizing deformation of the skin.

### SUMMARY OF THE INVENTION

The present invention is a microneedle device for delivery or sampling of fluids to or from intradermal layers of the skin of a mammalian subject.

According to the teachings of the present invention there is provided, a microneedle device for delivery or sampling of fluids to or from intradermal layers of the skin of a mammalian subject, the device comprising: (a) a skin contact configuration configured to contact an external surface of the skin so as to define a predefined orientation of the device relative to a reference plane corresponding to an initial position of the surface of the skin, (b) at least one microneedle having at least one peripheral surface converging to a tip, the microneedle being mechanically linked to the skin contact configuration so as to define an orientation of the microneedle relative to the reference plane in which a first region of the peripheral surface is deployed substantially parallel to the reference plane; and (c) a fluid flow bore intersecting the first region of the peripheral surface

According to a further feature of the present invention, the at least one peripheral surface includes a first substantially planar surface corresponding to the first region

According to a further feature of the present invention, the at least one peripheral surface includes second and third peripheral surfaces arranged so as to define together an upward-facing blade extending from a base of the microneedle to the pointed tip.

According to a further feature of the present invention, the defined orientation of the microneedle is such that the first region lies no higher than the reference plane.

According to a further feature of the present invention, the defined orientation of the microneedle is such that the region lies below the reference plane.

According to a further feature of the present invention, the skin contact configuration includes a flat surface for abutting the external surface of the skin.

According to a further feature of the present invention, the at least one microneedle is implemented as a linear array of a plurality of microneedles

According to a further feature of the present invention, the at least one microneedle is formed on a substrate, and wherein the at least one peripheral surface includes a peripheral surface standing substantially upright relative to a surface of the substrate.

According to a further feature of the present invention, the substrate and the at least one microneedle are integrally formed from a single crystal of material, the first region lying on an additional peripheral surface corresponding to a crystallographic plane of the single crystal.

According to a further feature of the present invention, the crystallographic plane is inclined relative to the surface of the substrate at an angle of about 54 7 degrees, and wherein the defined orientation of the microneedle is such that the surface of the substrate is inclined at an angle of between 50 and 60 decrees to the reference plane

According to a further feature of the present invention, the skin contact configuration includes a block providing a contact surface for abutting the external surface of the skin and a relief surface for attachment of the substrate, the block being formed with an internal angle of between 120 degrees and 130 degrees between the contact surface and the relief surface.

There is also provided according to the teachings of the present invention, a microneedle device for delivery or sampling of fluids to or from intradermal layers of the skin of a mammalian subject, the device comprising: (a) a microneedle arrangement including a linear array of microneedles projecting from a surface of a substrate, each of the microneedles having at least one peripheral surface standing substantially upright from the substrate surface and an inclined surface intersecting with the at least one upright surface to form a tapered shape terminating at a pointed tip, the inclined surface forming a first angle θ relative to the substrate; (b) a fluid flow bore intersecting the inclined surface; and (c) a block providing a contact surface for abutting the external surface of the skin and a relief surface for attachment of the substrate, the block being formed with an internal angle of substantially (180-θ) decrees between the contact surface and the relief surface such that the inclined surface is substantially parallel to the contact surface.

According to a further feature of the present invention, a base of the inclined surface of each microneedle is adjacent to an edge of the substrate, and wherein the substrate is attached to the block adjacent to a junction of the contact surface and the relief surface such that the inclined surface is deployed below the contact surface.

Accordingly to a further feature of the present invention, the inclined surface corresponds to a crystallographic plane inclined at an angle of about 54.7 degrees to the substrate surface

According to a further feature of the present invention, the at least one substantially upright peripheral surface includes two surfaces arranged so as to define together an upward-facing blade extending from the substrate surface to the pointed tip.

### BRIEF DESCRIPTION OF THE DRAWINGS

The invention is herein described, by way of example only, with reference to the accompanying drawings, wherein:

FIG. 1A, referred to above, is a schematic isometric view of a prior art microneedle device,

FIG. 1B is a side cross-sectional view of the device of FIG. 1A,

FIG. 2A, referred to above, is a schematic side cross-sectional view of a prior art microneedle device, corresponding to FIG. 11j of WO 2005/049107;

FIG. 2B is a schematic isometric view illustrating the deformation of skin over the microneedle of FIG. 1A when inserted into the skin;

FIG. 3A, referred to above, is a schematic side cross-sectional view of a prior art microneedle device, corresponding to FIG. 11h of WO 2005/049107;

FIG. 3B is a schematic isometric view illustrating the deformation of skin over the microneedle of FIG. 2A when inserted into the skin;

FIG. 4A is a schematic side cross-sectional view of a microneedle device, constructed and operative according to the teachings of the present invention, showing the geometry of the microneedle and skin contact surfaces of the device according to a first aspect of the present invention;

FIG. 4B is a schematic isometric view illustrating the deformation of the skin over the microneedle of FIG. 3A when inserted into the skin;

FIG. 5A is an isometric view of a microneedle device, constructed and operative according to the teachings of a second aspect of the present invention;

FIG. 5B is a schematic side cross-sectional view taken through the device of FIG. 5A,

FIG. 6A is an isometric view of an alternative microneedle device similar to the device of FIG. 5A implemented as a syringe adapter,

FIG. 6B is a schematic side cross-sectional view taken through the device of FIG. 6A;

FIG. 7A is an isometric view of a microneedle device, constructed and operative according to the teachings of the present invention, implemented as a syringe adapter employing a geometry similar to that of FIG. 4A,

FIG. 7B is a side view of the device of FIG. 7A;

FIG. 7C is an enlarged view of the microneedle region of FIG. 7B,

FIG. 7D is a partially cut-away view of the device of FIG. 7A;

FIG. 7E is an enlarged view of the region of microneedle attachment from FIG. 7D;

FIG. 8A is an isometric view of a preferred implementation of a microneedle for use in the device of FIG. 7A;

FIG. 8B is a plan view of the microneedle of FIG. 8A;

FIG. 8C is a side view of the microneedle of FIG. 8A;

FIG. 9A is a schematic side view of the device of FIG. 7A during initial anchoring into the skin of a mammalian subject;

FIG. 9B is a schematic side view of the device of FIG. 4 deployed for injection of fluid into intradermal layers of the skin,

FIGS. 10A and 10B are a plan view and an isometric view, respectively, of an alternative implementation of a microneedle, constructed and operative according to the teachings of the present invention, illustrating a fluid flow bore with an elliptical cross-sectional shape;

FIGS. 11A and 11B are a plan view and an isometric view, respectively, of an alternative implementation of a microneedle, constructed and operative according to the teachings of the present invention, illustrating a fluid flow bore with a triangular cross-sectional shape;

FIGS. 12A and 12B are a plan view and an isometric view, respectively, of an alternative implementation of a microneedle, constructed and operative according to the teachings of the present invention, illustrating a fluid flow bore with a pentagonal cross-sectional shape;

FIGS. 13A and 13B are a plan view and an isometric view, respectively, of an alternative implementation of a microneedle, constructed and operative according to the teachings of the present invention, illustrating a fluid flow bore with an alternative cross-sectional shape;

FIGS. 14A and 14B are a plan view and an isometric view, respectively, of an alternative implementation of a microneedle, constructed and operative according to the teachings of the present invention, having a curved uptight wall and illustrating a fluid flow bore with an elliptical cross-sectional shape;

FIGS. 15A and 15B are a plan view and an isometric view, respectively, of an alternative implementation of a microneedle, constructed and operative according to the teachings of the present invention, having a curved upright wall and illustrating a fluid flow bore with a correspondingly curved cross-sectional shape;

FIGS. 16A and 16B are a plan view and an isometric view, respectively, of an alternative implementation of a microneedle, constructed and operative according to the teachings of the present invention, having a curved upright wall and illustrating a fluid flow bore with an alternative cross-sectional shape,

FIGS. 17A and 17B are a plan view and an isometric view, respectively, of an alternative implementation of a microneedle, constructed and operative according to the teachings of the present invention, having a triangular external shape and illustrating a fluid flow bore with a triangular cross-sectional shape;

FIGS. 18A and 18B are a plan view and an isometric view, respectively, of an alternative implementation of a microneedle, constructed and operative according to the teachings of the present invention, having a triangular external shape and illustrating a fluid flow bore with a rounded pentagonal cross-sectional shape;

FIGS. 19A and 19B are a plan view and an isometric view, respectively, of an alternative implementation of a microneedle, constructed and operative according to the teachings of the present invention, having a triangular external shape and illustrating a fluid flow bore with an alternative cross-sectional shape; and

FIGS. 20A and 20B are a plan view and an isometric view, respectively, of an alternative implementation of a microneedle, constructed and operative according to the teachings of the present invention, having a triangular external shape and illustrating a fluid flow bore with an elliptical cross-sectional shape.

### DESCRIPTION OF THE PREFERRED EMBODIMENTS

The present invention is a microneedle device for delivery or sampling of fluids to or from intradermal layers of the skin of a mammalian subject.

The principles and operation of devices according to the present invention may be better understood with reference to the drawings and the accompanying description.

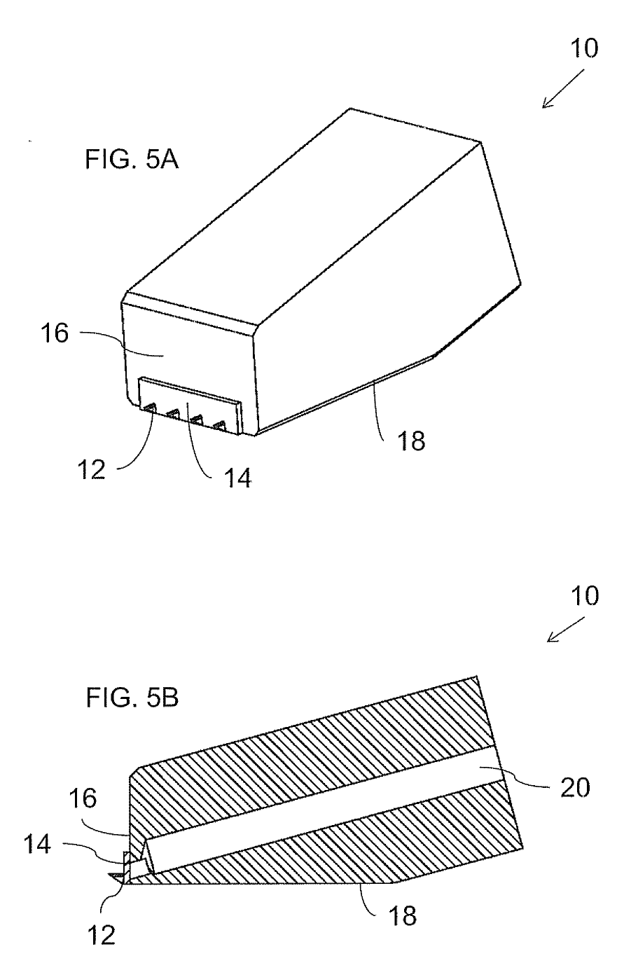

By way of introduction, it should be noted that the present invention provides two distinct aspects, each of which may be used alone to advantage, and which are most preferably combined in synergy to provide a particularly preferred implementation of the invention. Specifically, one aspect of the invention relates to the geometry of flow channels within a block, allowing the channels to reach a “corner” region of the block as required for the aforementioned “side insertion” technique and ensuring that the end of the flow channel away from the microneedle interface is conveniently positioned to allow attachment of a fluid supply device without complicating the fluid flow channels within the block This aspect of the present invention will be described particularly with reference to FIGS. 5A-7B.

A further aspect of the present invention relates to a particularly advantageous needle geometry which achieves particularly shallow penetration when used in the “side insertion” technique This aspect of the present invention will be described particularly with reference to FIGS. 4A-4B and 7A-9B.

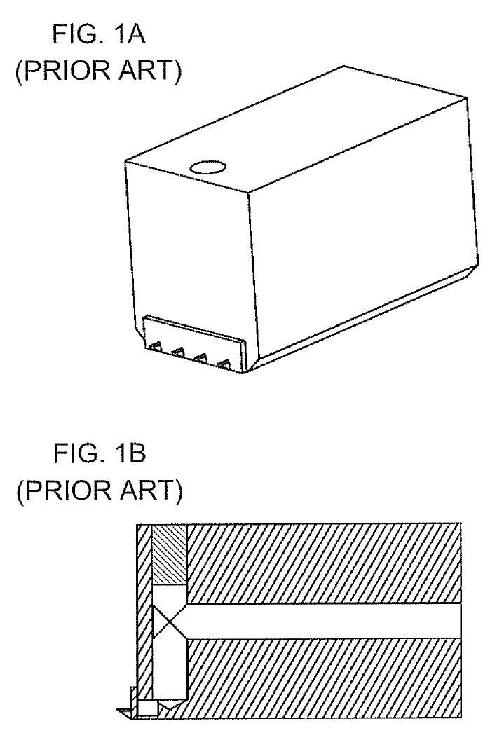

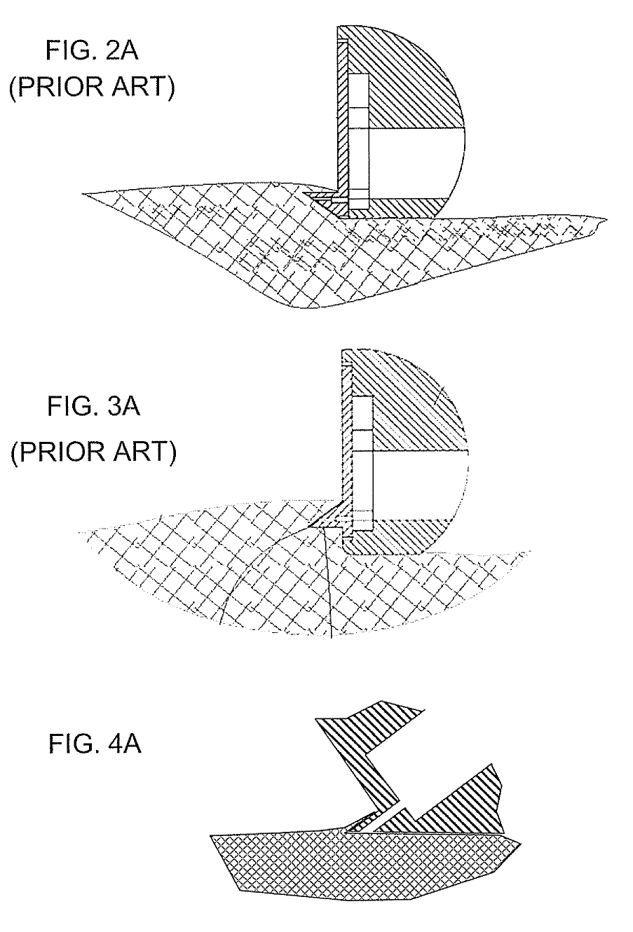

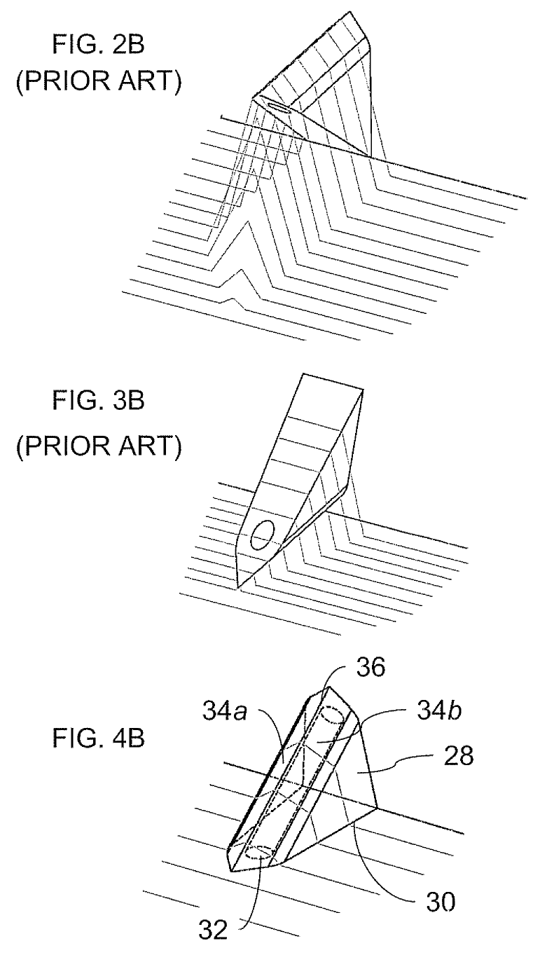

Referring now to the drawings, FIGS. 5A and 5B show an implementation of a microneedle device, generally designated 10, constructed and operative according to the teachings of the present invention, illustrating the flow channel aspect of the present invention. The geometry of the microneedle-skin interface of device 10 is essentially equivalent to that of the side insertion microneedle device of FIG. 1, including a linear array of microneedles 12 projecting from a substrate 14 attached to a relief surface 16 adjacent to a skin contact surface 18, and where the relief surface and skin contact surface are roughly orthogonal. Unlike the device of FIG. 1, however, a primary flow channel 20 passing through the block is inclined at an acute angle relative to skin contact surface, in this case around 15 degrees In the particularly preferred example illustrated here, at least some of the rear surfaces of the block are oriented in parallel and/or orthogonal relation to the direction of primary flow channel 20. The result may be viewed as a rectangular block with a central flow channel where skin contact surface 18 and relief surface 16 are formed by truncating the block at angles other than the orthogonal planes of the original block.

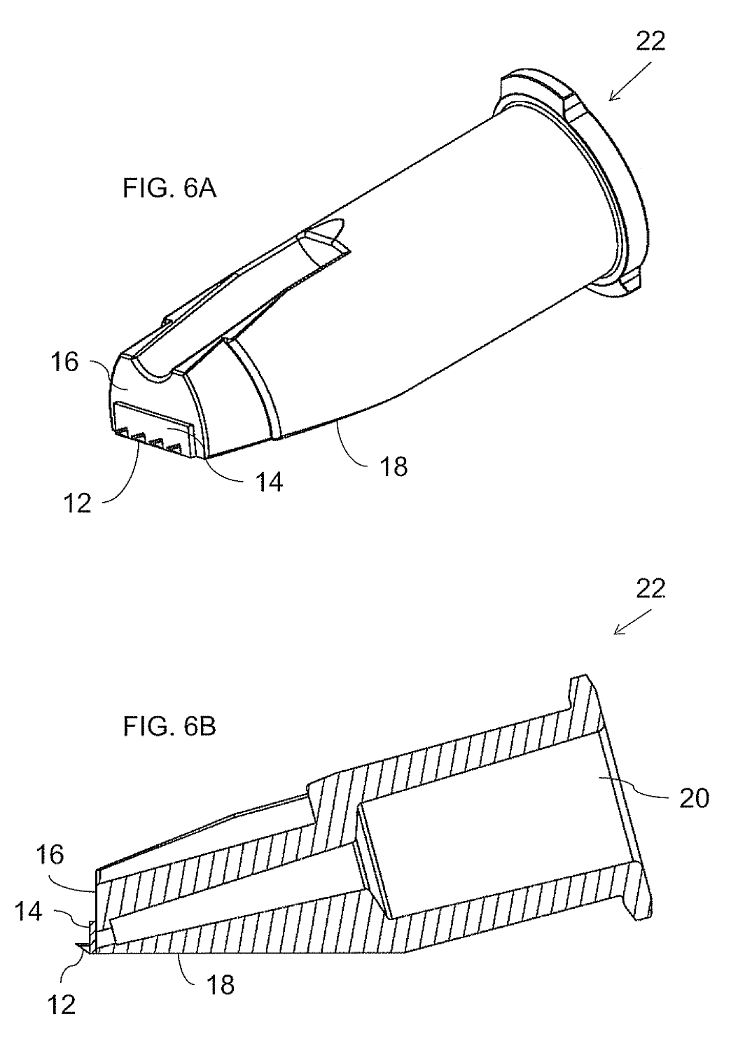

Turning now to FIGS. 6A and 6B, these show an implementation of a microneedle device, generally designated 22, which is generally similar to device 10 of FIGS. 5A and 5B, similar elements being designated similarly. Device 22 differs primarily in that the block is a molded unit configured to act as an adapter for attachment to a syringe, thus operating as a replacement for a conventional disposable needle. In the case shown here, flow channel 20 is formed with a slightly conical shape configured to provide a standard female luer connector for engagement on a syringe tip. Here, the angle of channel upwards away from the skin also facilitates correct positioning of the syringe. In order to optimize the design for plastic molding production techniques, the adapter is preferably implemented in a substantially cylindrical form to avoid extreme variations in wall thickness and to allow slowly varying wall thickness.

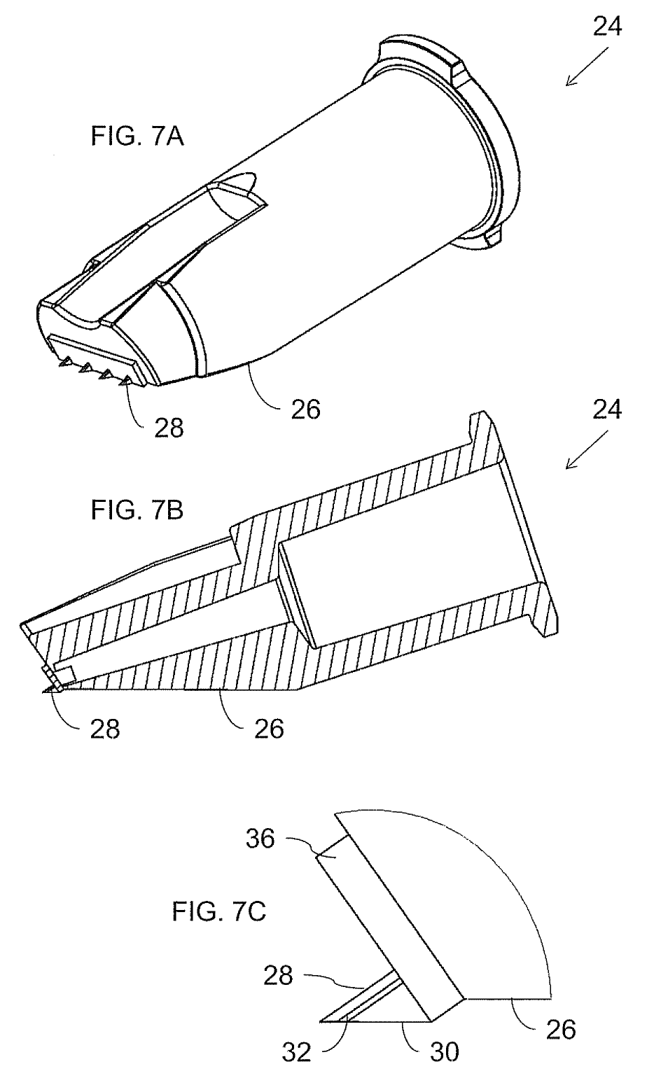

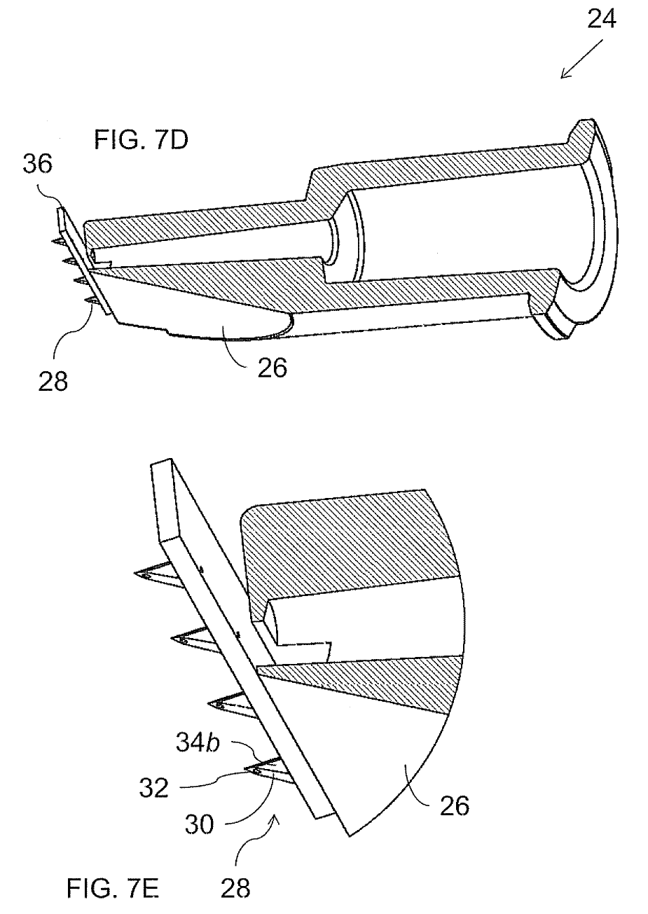

Turning now to FIGS. 7A-9B, these illustrate a further implementation of a microneedle device, generally designated 24, constructed and operative according to the teachings of the present invention. Microneedle device 24 is generally similar to device 22, but illustrates also a second aspect of the present invention, namely, a preferred geometry of microneedles as exemplified in FIGS. 4A and 4B

In general terms, microneedle device 24 includes a skin contact configuration configured to contact an external surface of the skin so as to define a predefined orientation of the device relative to a reference plane corresponding to an initial position of the surface of the skin. This skin contact configuration is most preferably implemented as a flat skin contact surface 26 in which case the reference plane corresponds to the plane of surface 26 Microneedle device 24 also includes at least one microneedle, and preferably a linear array of microneedles 28, each having at least one, and preferably several, peripheral surfaces converging to form a tapered shape terminating at a pointed tip Microneedles 28 are mechanically linked to the skin contact configuration so as to define an orientation of the microneedles relative to the reference plane in which a first of the peripheral surfaces 30, or at least a region thereof, is deployed substantially parallel to, i.e, within ±10 degrees, and more preferably within ±5 degrees, of the reference plane In certain particularly preferred implementations, surface 30 is deployed so as to be no higher than the reference plane. Each microneedle 28 is further formed with a fluid flow bore 32 intersecting first peripheral surface 30.

Before addressing the features of various specific implementations of the present invention in more detail, it will be useful to define certain terminology as used herein in the description and claims. Firstly, the device is described as delivering a fluid into a flexible biological barrier. While the invention may be used to advantage for delivery of fluids through a wide range of biological barriers including the walls of various internal organs, the invention is primarily intended for delivery of fluids into, or fluid sampling from, layers of the skin of a mammalian subject, and in particular, for intradermal or intra-epidermal delivery of fluids into the skin of a human subject. The fluids delivered may be any fluids. Preferred examples include, but are not limited to, dermatological treatments, vaccines, and other fluids used for cosmetic, therapeutic or diagnostic purposes. Furthermore, although considered of particular importance for intradermal fluid delivery, it should be noted that the present invention may also be applied to advantage in the context of transdermal fluid delivery and/or fluid aspiration such as for diagnostic sampling.

Reference is also made to geometrical relations to the surface of the flexible biological barrier. For the purpose of the present description and the appended claims, all geometrical relations to the “surface” of the flexible biological barrier are defined in relation to a plane approximating to the surface of the barrier in an initial state of rest of the biological barrier, i e., prior to any deformation of the barrier caused by insertion of the microneedle fluid delivery configuration. As a more technical definition, particularly important in the case of a region of skin which has considerable curvature, this surface is defined as the plane containing two orthogonal tangents to the flexible biological barrier surface at the location of interest.

For convenience, directions or positions further from the surface of the skin are referred to as “up”, “above” or other similar terms, and directions or positions closer to, or deeper within, the skin are referred to as “down”, “below” or other similar terms. It will be understood that this terminology is arbitrary in the sense that the skin surface itself may have any orientation in space.

Where reference is made to a direction of motion having a component parallel to the surface of the biological barrier, this includes any motion which is not perpendicular to the skin surface. Preferably, the motion has a majority component parallel to the skin surface, i.e, at an angle shallower than 45 decrees Most preferably, the part of the motion performed in contact with the skin is performed substantially parallel to the skin's surface, i e, with a motion vector not more than about ±15 degrees above or below the plane of the skin surface at rest

With regard to angles relative to the plane of the skin, angles will be referred to relative to a vector parallel to the skin as zero decrees with angles pointing into the skin being positive and angles away (outwards) from the skin being designated negative. For simplicity of presentation use may be made of the term “upwards” or “up” to refer to directions outwards from the initial plane of the skin and “downwards” or “down” to refer to directions inwards or towards the initial plane of the skin

Reference is also made to various physical states of the biological barrier. The biological barrier is described as “stretched” when a distance between points defined on the barrier in at least one direction is greater than the distance between the same two points when the skin is released. The direction of maximum strain is referred to simply as the stretching direction “Unstretched” denotes a state of the skin where no stretching is present parallel to the direction of stretching in an adjacent region of stretched skin. It will be appreciated that, where compression of skin tissue has lead to local bulging or folding of the tissue, a degree of stretching may occur perpendicular to the compression vector to accommodate the out-of-plane distortion of the tissue.

Nevertheless, such tissue is referred to herein as “unstretched” since no elongation is present in the direction of stretching. Tissue for which the distance between points is reduced relative to the same two points when the skin is released is referred to as “relaxed” tissue since it exhibits lower surface tension than the skin when released.

The present invention is referred to as employing one or more microneedle The term “microneedle” is used herein in the description and claims to refer to a structure projecting from an underlying surface to a height of no more than 1 mm, and preferably having a height in the range of 50 to 500 microns. The microneedles employed by the present invention are preferably hollow microneedles having a fluid flow channel formed therethrough for delivery of fluid The height of the microneedles is defined as the elevation of the microneedle tip measured perpendicularly from the plane of the underlying surface. The term “peripheral surface” is used to refer to any surface of the microneedle which is not parallel to the surrounding substrate surface. The term “upright” surface is used to refer to any surface which stands roughly perpendicular to the surrounding substrate surface.

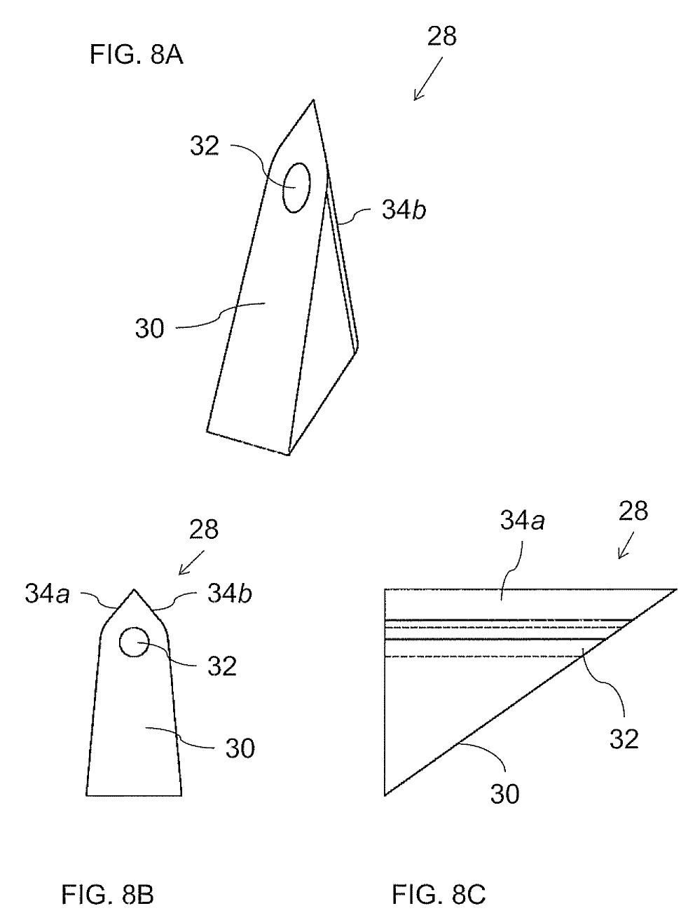

As mentioned above, most preferred implementations of the present invention employ microneedles of a type similar to those disclosed in co-assigned U.S. Pat. No. 6,533,949, namely, formed with at least one wall standing substantially perpendicular to the underlying surface and deployed so as to define an open shape as viewed from above, the open shape having an included area, and an inclined surface inclined so as to intersect with the at least one wall, the intersection of the inclined surface with the at least one wall defining at least one cutting edge. The fluid flow channel is preferably implemented as a bore intersecting with the inclined surface. The particular robustness of the aforementioned microneedle structure and its particular geometrical properties exhibit great synergy with the structures and insertion methods of the present invention, ensuring that the microneedles can withstand the applied shear forces and are optimally oriented for delivery of fluids into the biological barrier These advantages with be detailed further below One particularly preferred microneedle structure, and corresponding preferred ranges of parameters for microneedles of the present invention, will be described below with reference to FIGS. 8A-8C.

Reference is also made to various surfaces which may be provided by a “block of material”. The term “block” is used herein to refer generically to any structure of one unitary element or plural elements cooperating to provide the recited surfaces in fixed mechanical relation The “block” thus described includes, but is not limited to, a solid block, a hollow block, a thin sheet-like block and an open arrangement of surfaces mechanically interconnected to function together as a block Part or all of the block may also be provided by a substrate upon which the microneedles are integrally formed.

The present invention relates to a “fluid transfer interface”, i.e., the structure and the operation of a microneedle arrangement which interfaces with the biological barrier to create a fluid transfer (delivery or sampling) path into or out through the barrier The fluid transfer interface may be integrated as part of a self-contained fluid delivery device, or as an adapter device for use with an external fluid supply device The term “fluid” is used to refer to any composition which flows, or can be induced to flow under working conditions of the device Thus defined, “fluid” includes, but is not limited to, any and all types of liquid, gel, suspension or fluidized powder.

Referring specifically to FIGS. 4A, 4B and 8A-8C, a particularly preferred implementation of microneedles 28 has second and third peripheral surfaces 34a and 34b arranged so as to define together an upward-facing, blade 36 extending from a base of microneedle 28 to its pointed tip As best seen in FIG. 4B, the resulting microneedle structure has particular advantages in minimizing deformation of the skin. Firstly, the downward slope of the blade 36 towards its tip ensures that the outermost layers of the skin are minimally stretched near the thinner tip portion of the needles Furthermore, blade 36 is effective to define a parting line at which the upper skin layers are cut as they near the base of the microneedle, thereby ensuring that the microneedle remains inserted as shallowly as possible in the skin, and even cuts its way upwards through any overlying layers of soft tissue if it was initially made to penetrate more deeply. Complete egress of the needles from the skin is typically prevented by the relative hardness of the upper layer of the skin (stratum corneum) in combination with the low tension applied by the microneedle near its thin tip The deployment of downward-facing surface 30 with its fluid flow bore 32 parallel to the skin and at a depth very close to the surface of the skin ensures that any fluid delivery or sampling occurs very shallowly. Production techniques suitable for producing this preferred microneedle structure will be fully understood by one ordinarily skilled in the art by reference to the aforementioned U.S. Pat. No. 6,533,949

According to a particularly preferred implementation, it has been found advantageous to use microneedles having a height of between 300 and 500 microns, and most preferably about 450±20 microns In order to provide an effective cutting edge 36 while leaving sufficient space for a fluid flow bore 32 relatively high up the microneedle, peripheral surfaces 34a and 34b preferably form between them an angle of between about 65° and about 80°. This facilitates use of a fluid flow bore of diameter 30-60 microns, and most preferably 45±5 microns. Preferably, bore 32 is positioned so as to leave a minimum wall thickness of at least about 30 microns

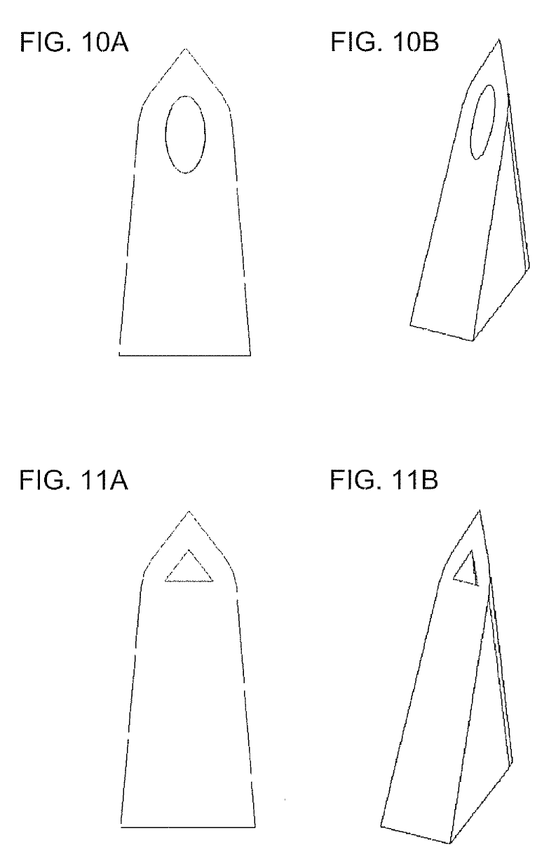

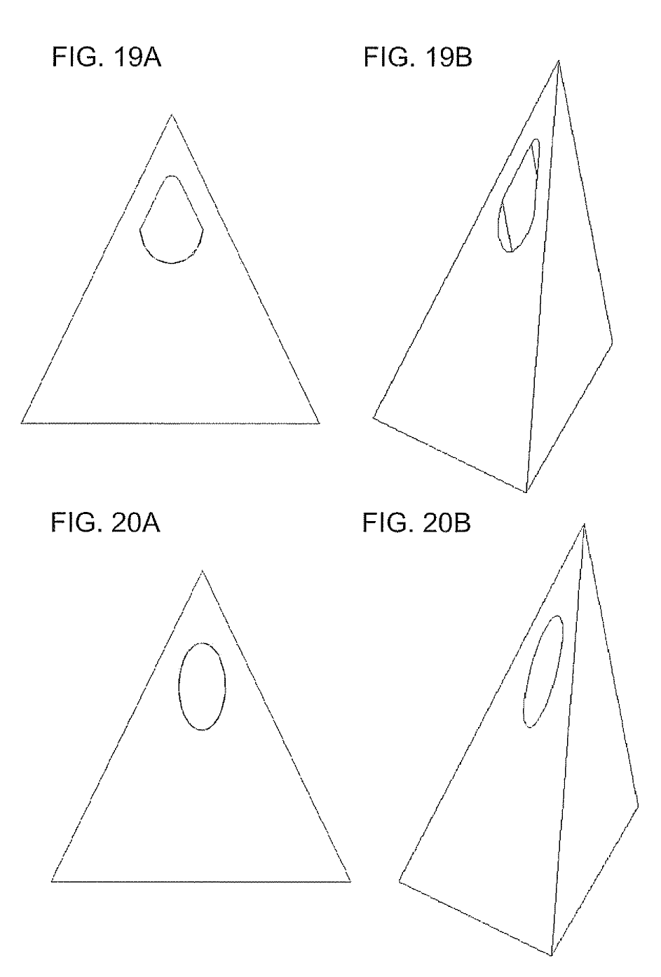

Referring parenthetically to FIGS. 10A-20B, it will be noted that various geometrical parameters of the microneedles of the present invention may vary considerably. In particular, it should be noted that the shape of the fluid flow bore of microneedles according to the present invention is not necessarily, or even typically, round. The dry etching process used to define the shape of the bore allows great flexibility regarding the shape of the bore The preferred shape is typically dictated by one or more factors including, but not limited to a minimum wall thickness between the bore and the peripheral walls to ensure structural integrity of the microneedles, the height of the opening of the bore relative to the total height of the microneedle, starting at a height sufficient to ensure non-leaking fluid transfer and extending close enough to the penetrating tip to avoid unnecessarily deep penetration for shallow delivery, and the total cross-sectional area of the bore sufficient to deliver the desired flow rates.

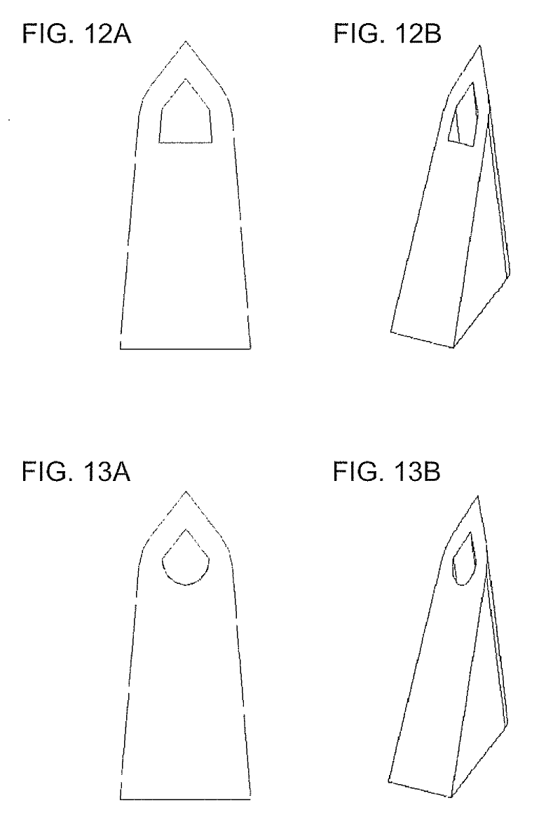

By way of a number of non-limiting preferred examples, the bore area may be enlarged without getting closer to the peripheral walls by using an elliptical shape as illustrated in FIGS. 10A and 10B. In order to maximize the area while keeping the bore as close to the microneedle tip as possible, a triangular bore shape as shown in FIGS. 11A and 11B may be used Where a larger flow area is required, the triangle may be supplemented to give a pentagonal form as in FIGS. 12A and 12B, or by a rounded opening as shown in FIGS. 13A and 13B.

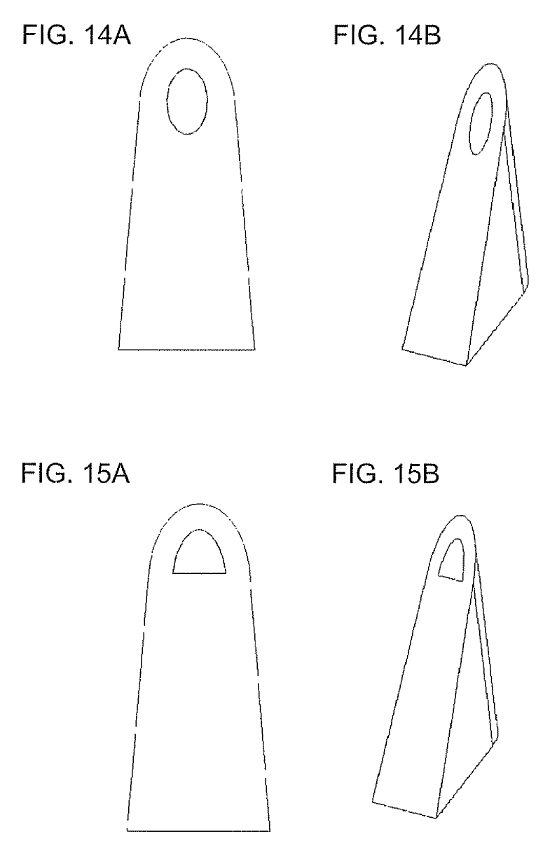

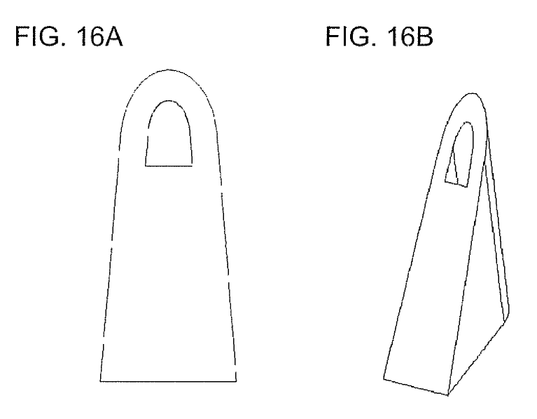

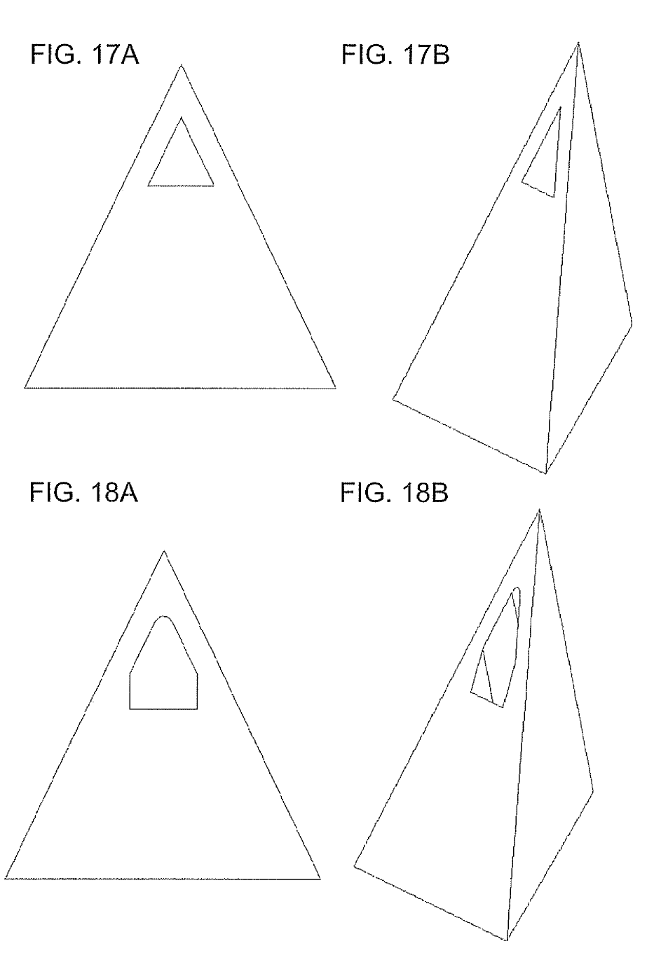

Although the pentagonal outline of the microneedles of FIG. 5A-5C and 10A-13B are believed to be particularly advantageous for the present invention, it should be noted that similar families of implementations of microneedles with different bore shapes may be produced in microneedles of other external shapes By way of example, FIGS. 14A-16B illustrate various microneedles with rounded upright walls showing bore shapes which are elliptical (FIGS. 14A and 14B), rounded parallel to the rounded outer wall (FIGS. 15A and 15B), and similarly rounded but further extended towards the base (FIGS. 16A and 16B). Similarly, in the context of a microneedle of triangular outer shape, FIGS. 17A-20B show, in respective pairs, implementations with bores formed as triangles, rounded pentagons, rounded triangles with a semicircular base, and an ellipse. In all of the above cases, it should be noted that the cross-sectional shape of the bore need not necessarily be uniform through the entire thickness of the microneedle and substrate

Referring now particularly to FIGS. 7C and 7E, there is shown a preferred implementation of attachment of microneedles 28. Specifically, microneedles 28 are preferably formed on a substrate 38 which typically has a thickness no more than about 0.3 mm, and most preferably about 0 2 mm when a 0.65 mm wafer is used. The microneedles are preferably formed with surfaces 34a and 34b standing upright (roughly perpendicular) to the substrate surface and surface 30 inclined so as to intersect the upright surfaces In a preferred case of microneedles formed from a single crystal of material such as silicon, surface 30 is preferably a crystallographic plane of the crystal from which the needles and substrate are formed Using Miller indices, most preferably, the substrate surface is a typical (100) plane and surface 30 is a typical (111) plane, giving an inclination angle of about 54 7 degrees between surface 30 and the plane of the substrate. Other implementations are also possible, for example, employing the (221) plane giving an inclination of about 48 2 degrees, employing the (211) plane giving an inclination of about 35 3 degrees, the (311) plane giving an inclination of about 25.2 degrees, the (122) plane giving an inclination of about 70.5 degrees, or the (133) plane giving an inclination of about 76.7 degrees.

In order to ensure that surface 30 is substantially parallel to skin contact surface 28, substrate 38 is preferably mounted on a relief surface 40 which is inclined at a roughly corresponding angle relative to the reference plane In the preferred example of a (111) crystallographic plane, relief surface 40 is preferably inclined upwards relative to the reference plane at an angle of between 50 and 60 degrees to the reference plane, and most preferably around 55 degrees. In a preferred case where skin contact surface 26 and relief surface 40 are provided by faces of a single block, the block is therefore formed with an internal angle of between 120 decrees and 130 decrees between contact surface 26 and relief surface 40. In more general terms, where the angle of inclination of inclined surface 30 to the surface of substrate 36 is θ, the internal angle of the block is preferably substantially (180-θ) degrees so that surface 30 ends up substantially parallel to the skin contact surface 26

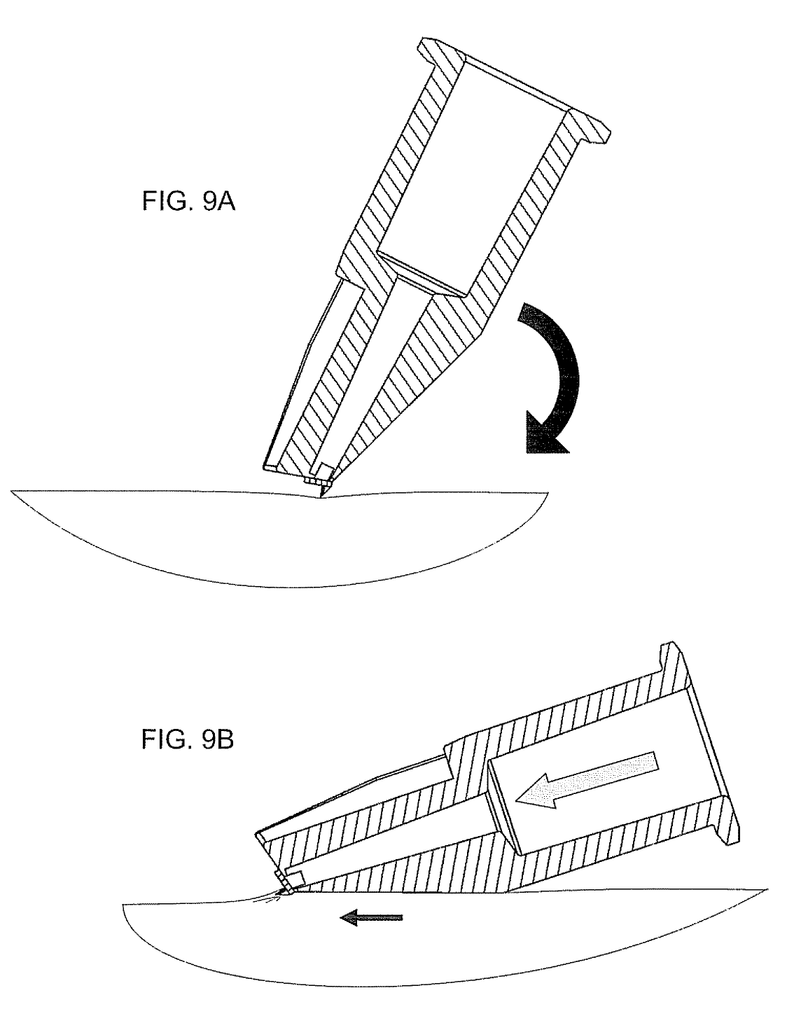

As best seen in FIG. 7C, microneedles 28 are preferably deployed with surfaces 30 terminating adjacent to the lower edge of substrate 38. The thickness of substrate 38 preferably generates a small downwards step such that the plane of surface 30 is deployed slightly below the plane of skin contact surface 26. This geometry also helps to prevent egress of the microneedles from the skin as the device is pushed parallel to the skin in a direction as shown by the arrow in FIG. 9B

Although the present invention has been described herein with reference to a preferred implementation employing silicon microneedles, it should be noted that the invention is not limited to such implementations and may alternatively be implemented using a wide range of other materials. Suitable examples include, but are not limited to polymer microneedles formed, for example, by microinjection molding; microneedles formed from radiation-sensitive polymers such as by the techniques described in co-assigned U.S. Pat. No. 6,924,0874; and metal foil implementations using microneedles formed by stamping techniques, all as known to one ordinarily skilled in the art.

Furthermore, it will be noted that the form of microneedles used to implement the present invention may be any form which satisfies the geometrical requirements stated above, and may vary considerably from the preferred micro-pyramid form described. Thus, by way of non-limiting examples, suitable forms of microneedles include: conical microneedles with an asymmetric fluid flow bore; and pyramidal microneedles structures with various polygonal base shapes, such as a hexagonal base, with an asymmetric fluid flow bore. In the case of a conical needle, the region parallel to the reference plane is preferably the region lying along the bottom edge of the conical shape.

Turning finally to FIGS. 9A and 9B, these illustrate the operation of the present invention, which is essentially similar to that of the side insertion devices of the aforementioned WO 2005/049107 A2 and US 2005/0209566, and will best be understood by analogy therewith. Specifically, as shown in FIG. 9A, the device is preferably first anchored into the skin by gentle pressure of the needles while being held at an elevated angle. Then, as shown in FIG. 9B, the device is brought into its normal operative relation to the skin and gently displaced relative to the skin in the direction of the arrow As mentioned above, the preferred geometry of microneedles 28 helps to bring the needles to a shallow position within the skin layers, and provides low flow resistance to injection of fluid via the downward-facing, fluid flow bore 32

According to a further option, it should be noted that the structures of the present invention may be used to advantage for a process of high-pressure injection of fluids into the body For example, using a normal syringe, injection may be performed at a pressure of between about 100 and about 1000 PSI In certain preferred applications, a low-volume precision syringe, such as a HAMILTON® syringe, can be used to generate injection pressures in the range of 1000-4000 PSI. These pressures may be effective to enhance penetration and/or dispersion of the injected fluid into tissue due to mechanical action of the resulting “jet” of fluid.

It will be appreciated that the above descriptions are intended only to serve as examples, and that many other embodiments are possible within the scope of the present invention as defined in the appended claims

What is claimed is:

1. A microneedle device for delivery or sampling of fluids to or from intradermal layers of the skin of a mammalian subject, the device comprising:
(a) a skin contact configuration configured to contact an external surface of the skin so as to define a predefined orientation of the device relative to a reference plane corresponding to an initial position of the surface of the skin;
(b) at least one microneedle having at least one peripheral surface converging to a tip, said microneedle being mechanically linked to said skin contact configuration so as to define an orientation of said microneedle relative to said reference plane in which a first region of said peripheral surface is deployed substantially parallel to said reference plane; and
(c) a fluid flow bore intersecting said first region of said peripheral surface.

2. The device of claim 1, wherein said at least one peripheral surface includes a first substantially planar surface corresponding to said first region.

3. The device of claim 1, wherein said at least one peripheral surface includes second and third peripheral surfaces arranged so as to define together an upward-facing blade extending from a base of said microneedle to said pointed tip.

4. The device of claim 1, wherein said defined orientation of said microneedle is such that said first region lies no higher than said reference plane.

5. The device of claim 1, wherein said defined orientation of said microneedle is such that said region lies below said reference plane

6. The device of claim 1, wherein said skin contact configuration includes a flat surface for abutting the external surface of the skin.

7. The device of claim 1, wherein said at least one microneedle is implemented as a linear array of a plurality of microneedles.

8. The device of claim 1, wherein said at least one microneedle is formed on a substrate, and wherein said at least one peripheral surface includes a peripheral surface standing substantially upright relative to a surface of said substrate.

9. The device of claim 8, wherein said substrate and said at least one microneedle are integrally formed from a single crystal of material, said first region lying on an additional peripheral surface corresponding to a crystallographic plane of the single crystal.

10. The device of claim 9, wherein said crystallographic plane is inclined relative to said surface of said substrate at an angle of about 54 7 degrees, and wherein said defined orientation of said microneedle is such that said surface of said substrate is inclined at an angle of between 50 and 60 degrees to said reference plane.

11. The device of claim 10, wherein said skin contact configuration includes a block providing a contact surface for abutting the external surface of the skin and a relief surface for attachment of said substrate, said block being formed with an internal angle of between 120 degrees and 130 degrees between said contact surface and said relief surface

12. A microneedle device for delivery or sampling of fluids to or from intradermal layers of the skin of a mammalian subject, the device comprising:
(a) a microneedle arrangement including a linear array of microneedles projecting from a surface of a substrate, each of said microneedles having at least one peripheral surface standing substantially upright from said substrate surface and an inclined surface intersecting with said at least one upright surface to form a tapered shape terminating at a pointed tip, said inclined surface forming a first angle θ relative to said substrate,
(b) a fluid flow bore intersecting said inclined surface, and
(c) a block providing a contact surface for abutting the external surface of the skill and a relief surface for attachment of said substrate, said block being formed with an internal angle of substantially (180-θ) degrees between said contact surface and said relief surface such that said inclined surface is substantially parallel to said contact surface.

13. The device of claim 12, wherein a base of said inclined surface of each microneedle is adjacent to an edge of said substrate, and wherein said substrate is attached to said block adjacent to a junction of said contact surface and said relief surface such that said inclined surface is deployed below said contact surface.

14. The device of claim 12, wherein said inclined surface corresponds to a crystallographic plane inclined at an angle of about 54 7 degrees said substrate surface.

15. The device of claim 12, wherein said at least one substantially upright peripheral surface includes two surfaces arranged so as to define together an upward-facing blade extending from said substrate surface to said pointed tip.
